(19) United States
(12) Patent Application Publication
(10) Pub. No.: US 2026/0219146 A1
(43) Pub. Date: Jul. 30, 2026

(54) Sample Digestion for Mass Spectrometry

(71) Applicant: The Rosalind Franklin Institute, Oxfordshire (GB)

(72) Inventors: Zoltan Takats, Oxfordshire (GB); Daniel McGill, Oxfordshire (GB); Daniel Simon, Oxfordshire (GB)

(21) Appl. No.: 19/493,101

(22) PCT Filed: Jun. 12, 2024
(86) PCT No.: PCT/GB2024/051498

§ 371 Date: Dec. 12, 2025

Related U.S. Application Data
(63) Continuation-in-part of application No. PCT/GB2023/051572, filed on Jun. 15, 2023.

(30) Foreign Application Priority Data
Jun. 15, 2023 (WO) PCT/GB2023/051572

(51) Int. Cl. G01N 1/40; G01N 33/68; G01N 33/92; H01J 49/04; H01J 49/16

(52) U.S. Cl. CPC G01N 1/4044; G01N 33/6848; G01N 33/92; H01J 49/0431; H01J 49/168; G01N 2001/4038

(57) ABSTRACT

Apparatus and methods for analysing organic molecules of a condensed phase sample are disclosed. Such a method may comprise generating a plasma using an electrical discharge, carrying out a plasma treatment of the condensed phase sample by exposing the condensed phase sample to the plasma so as to generate fragments of the organic molecules within the condensed phase sample, and following completing the plasma treatment, using a mass spectrometer of other analysis device to detect the fragments, and characterising the fragments, the molecules or the sample using the detected fragments. The fragments may in particular arise from backbone fragmentation of the organic molecules.

The invention relates to the digestion of samples for subsequent mass spectrometry, and in particular to such digestion taking place within a condensed phase sample, for example prior to any subsequent desorption of components of the sample for introduction into a mass spectrometer for analysis. For example the digestion may be digestion of organic molecules within such condensed phase samples. The invention also relates to analysing such samples by carrying out such digestion or fragmentation to generate molecular fragments within the condensed phase sample, and then carrying out subsequent mass spectrometry, for example including desorption or ionisation, to detect the fragments.

### INTRODUCTION

Identification of proteins, peptides and other organic molecules, especially organic polymer molecules can be carried out using “top-down” whole molecule mass spectrometry, but this can be a challenging technique to use, particularly for proteins, and requires specific mass spectrometry instruments to carry out. “Bottom-up” mass spectrometry of corresponding molecules can be carried out instead, but typically involves using enzymatic digestion of such molecules, which is time consuming and requires use of reagents in the liquid phase. Such enzymatic digestion techniques also tend to lead to delocalisation of analytes in the sample so that structure and spatial resolution is lost.

It would be desirable to address these and other problems of the related prior art.

### SUMMARY OF THE INVENTION

The invention provides methods for analysis of samples by mass spectrometry or other analysis techniques, and in particular of organic molecule samples such as peptides and proteins, using a “bottom-up” approach, but without requiring enzymatic digestion which can be time consuming and require various liquid phase reagents to carry out. Instead a plasma pre-treatment or digestion process is completed on the condensed phase sample prior to any subsequent desorption or other process for introducing fragments of the sample generated during the plasma digestion process into a mass spectrometer or other type of analyser. Typically, the pre-treatment, plasma digestion process will take place and be completed at a first location, and the sample will then be transferred after completion of the pre-treatment to a second location for desorption and/or ionisation and/or other processes as part of the process of introducing the generated fragments into the mass spectrometer or other type of analyser. For example, introduction into the mass spectrometer or other type of analyser might take place at least one second, at least five seconds, at least ten seconds, at least thirty seconds, at least one minute, at least one hour, or at least one day after completion of the plasma digestion process.

Other suitable analysis methods and corresponding analysers for analysing the sample after the plasma digestion process include: nuclear magnetic resonance NMR or electron paramagnetic resonance EPR spectroscopy methods and analysers; and various optical spectroscopic methods and analysers for example operating in the infrared region, in the UV and/or visible regions, and using Raman spectroscopy, absorption and other techniques.

In particular, the invention provides methods and apparatus for analysing a condensed phase sample by using an electrical discharge or similar to complete a pre-treatment stage in which plasma fragmentation and digestion is completed within the condensed phase sample, for example without desorbing the sample at that time, and then subsequently, following completion of the pre-treatment, using mass spectrometry or another type of analysis of at least a portion of the sample comprising the generated fragments.

The target molecules for fragmentation in the sample may in particular be organic molecules, such as proteins or peptides, and especially organic polymer molecules. The described techniques are then particularly advantageous in generating for detection fragments of the organic molecules arising from fragmentation along the polymer backbone.

Desorption or similar processing of the sample for introduction into the mass spectrometer or other analysis device preferably takes place only after the plasma fragmentation and digestion process has taken place, and the two processes may typically take place at different locations or in different pieces of laboratory equipment, and for example at different times.

The plasma for causing fragmentation and digestion within the condensed phase sample may be generated within an environment of a suitable gas such as argon, to generate suitable fragments of the organic molecules, typically of sizes less than about 1000-2000 m/z. The technique can be regioselective in favouring the formation of particular fragments depending for example on conditions of the treatment such as the overall treatment period and electrical powers used to form the plasma, and the inventors' experimental evidence indeed shows that the cleavage of bonds within the organic molecules is reasonably consistent for a given organic molecule within the sample, the sample environment, and the plasma generation conditions. Because there is no need for addition of additional liquids such as enzyme solutions to achieve the fragmentation, solid samples can be treated in this way without delocalisation or movement of the target molecules for fragmentation within the sample.

After the plasma treatment of the condensed phase sample has been completed, which may for example be at least one second, at least five seconds, at least ten seconds, at least 30 seconds, at least one minute, at least one hour, or at least one day later, the resulting molecule fragments can then be analysed using known analysis methods such as mass spectrometry input and mass/charge separation methods, for example using direct infusion, liquid chromatography-mass spectrometry, or an imaging mass spectrometry modality, or other analysis techniques as mentioned above. Data resulting from the mass spectrometry or other analysis technique can then be used, for example, to determine if a particular protein or other molecule was present in the sample to which the plasma treatment was applied, or to carry out de novo sequencing or structural elucidation.

According to some aspects, the invention provides methods of analysing molecules of a condensed phase sample, comprising: generating a plasma for example using an electrical discharge; exposing the condensed phase sample to the plasma so as to generate fragments of the molecules within the condensed phase sample; and detecting the fragments using a mass spectrometer or other analysis device or apparatus. The fragments themselves or the molecules or the sample more generally may then be characterised, identified, or otherwise analysed using data from the mass spectrometer or other analysis device or apparatus relating to or including detection of the fragments.

Typically, the plasma treatment of the sample is carried out outside of, or away from, the mass spectrometer or other analysis device, and the treated sample is then transferred to and received by the mass spectrometer or other analysis device (or in particular an input part of the analysis device such as an ion source) for characterisation. The generated plasma used in the described techniques may be described as a cold plasma, a nonthermal plasma, or a low-temperature plasma. In such a plasma the electron temperature distribution is typically much hotter than the temperature of heavier ion and neutral species.

The condensed phase sample may be located on a solid substrate while being exposed to the plasma, for example on a microscope slide, or other similar substrate. In some arrangements the substrate may be provided with apertures for example being in the form of a mesh, which may assist in permitting the gas flow to bring the plasma into better contact with the sample. In some embodiments the condensed phase sample may be provided on or as part of an enzyme-linked immunosorbent assay (ELISA), a lateral flow test device, or any of various other types of assays and test devices, which will typically then provide the substrate on or in which the sample is disposed.

The sample may in particular comprise organic molecules, such as one or more of: proteins, peptides, lipids, other organic polymer molecules, and small molecules. Where the sample comprises organic polymer molecules, the described techniques are particularly advantageous in fragmenting the organic molecules along the polymer backbone. The condensed phase sample may be in liquid form, in solid form, or in some mixture of the two, but the described technique using plasma treatment is particularly advantageous in being applicable to solid samples.

Following exposure to the plasma, at least a portion of the condensed phase sample may be removed from the substrate, for example by mechanical removal in the existing condensed phase form, or by dissolving some of the sample in a solvent, and introduced to the mass spectrometer for subsequent ionisation and characterisation.

The described plasma treatment, including the electrical discharge, the plasma, and the exposed sample may typically take place at or close to ambient atmospheric pressure, for example between about 30 and 300 kPa.

The electrical discharge may be generated by applying an electrical signal to one or more electrodes, and examples of both AC and DC signals are described herein, but with AC signals generally being found by the inventors to provide more effective fragmentation of the condensed phase samples, and in particular radiofrequency signals. If an AC or radiofrequency signal is used then this may typically have a power of between 10 and 300 Watts, and/or a peak to peak voltage of between 1000 and 10000 V, and/or a frequency of from 100 KHz to 5 MHz.

The one or more electrodes may comprise a plasma source electrode arranged to generate the plasma between the plasma source electrode and the sample, and a ground plane electrode disposed on an opposite side of the sample from the plasma source electrode. For example, the ground plane electrode is may be disposed directly underneath the sample, or may be separated from the sample by some or all of the thickness of the substrate. In other embodiments, all electrodes may be provided on the same side of the sample and the generated plasma.

The method may also comprise providing a flow of a gas at the electrical discharge or from the electrical discharge towards or to the sample. Various such gases may be used, but may comprise at least 50%, or at least 90%, of one or more of: a noble gas, argon, helium, and nitrogen, with the inventors finding use of argon to be particularly effective. The gas may also contain one or more dopants, such as fluoranthene, at much lower levels of concentration, to support the above and other functions.

One of the one or more electrodes, for example the above mentioned plasma source electrode, may be provided by a conductor such as a needle extending along a conduit to an open end of the conduit, where it may protrude slightly from the conduit for example by between 0 and 5 mm. In this way the electrical discharge can be caused to take place proximally to the end of the conduit. The flow of the gas can then also be directed along the conduit.

The condensed phase sample may be exposed to the plasma for a suitable treatment period which can be determined or optimized experimentally, but according to experimentation already carried out by the inventors could for example be one or more of: at least 0.1 seconds, at least 1 second, at least 10 seconds, and at least 60 seconds.

The method may further comprise, before the step of exposing the condensed phase sample to the plasma so as to generate fragments of the organic molecules, treating the condensed phase sample with various compounds or reagents, for example a compound selected from a spin trap and a spin scavenger. This embodiment may be particularly advantageous when the electrical signal is a DC signal. Without wishing to be bound by theory, it is believed that when a DC signal is used to generate the plasma under the conditions described herein, at least some of the fragments generated from the organic molecules may be in the form of short-lived radicals. The spin trap or spin scavenger may react with these short-lived radicals to form stabilised species that can then be characterised using the mass spectrometer.

The invention also provides corresponding apparatus for treating a condensed phase sample for subsequent analysis, for example treating organic molecules of the condensed phase sample. Such apparatus may comprise: a plasma generator arranged to use an electrical discharge to generate a plasma; and a substrate carrier for receiving a substrate carrying the condensed phase sample such that the condensed phase sample can be exposed to the generated plasma, such that the plasma generates fragments of the organic molecules within the condensed phase sample.

Such apparatus may also comprise a mass spectrometer or other analysis device arranged to receive and detect the generated fragments after completion of the plasma treatment, and optionally a sample conveyor mechanism arranged to carry some or all of the condensed phase sample to the mass spectrometer or other analysis device following completion of the plasma treatment. The apparatus may also comprise the substrate and optionally the condensed phase sample itself, typically located on or in the substrate. Typically, the mass spectrometer or other analysis device is arranged to receive the already generated fragments from outside of the analysis device.

The apparatus, for example using a computer system of the apparatus, may then also be arranged to characterise or identify the sample, or more particularly the molecules or the fragments of the molecules themselves, using data from the mass spectrometer or other analysis device which includes data representing the detected fragments, for example specific data relating the fragments, or a mass spectrum, or optical spectral data, or other data within which the fragments are represented.

The substrate may be a solid substrate, for example a microscope slide, an insulating substrate, or a substrate carrying one or more electrodes, for example one or more ground plane electrodes as mentioned above, to provide a return current path for an electrical signal used in generating the plasma. The apparatus may further comprise a signal generator arranged to deliver an AC or DC electrical signal to the plasma generator for generating the plasma.

If an AC signal is used then this may one or more of: have a power of between 10 and 300 Watts; have a peak to peak voltage of between 1000 and 10000 V; and have a frequency of from 100 kHz to 5 MHz.

The apparatus may be further arranged to provide a flow of a gas at or past the electrical discharge, and optionally to the sample. The plasma generator may comprise one or more electrodes to which the AC or DC signal is applied in order to generate the electrical discharge. At least one of the one or more electrodes, for example one or more plasma source electrodes as mentioned above, may be provided by a conductor extending along a conduit to an open end of the conduit, so that the electrical discharge takes place proximally to the end of the conduit, and the flow of the gas is along the conduit.

Although the described methods and apparatus may use mass spectrometry as described in detail below to detect the fragments and characterise or identify the fragments, molecules, and/or the sample, other analysis techniques and instruments may be used instead or in addition to mass spectrometry as noted above, such as nuclear magnetic resonance spectroscopy or electron paramagnetic resonance spectroscopy and corresponding instruments.

### BRIEF DESCRIPTION OF THE DRAWINGS

Embodiments of the invention will now be described, by way of example only, with reference to drawings, of which:

FIG. 1 schematically shows apparatus for carrying out plasma treatment digestion of a sample to generate molecular fragments, and for subsequent detection of the fragments using a mass spectrometer or other analysis device or apparatus;

FIG. 2 illustrates a method according to the invention which can be carried out using the apparatus of claim 1;

FIG. 3 shows mass spectrometer measured intensity of various digest products of leucine enkephalin following the described plasma treatment over a variety of lengths of treatment periods;

FIG. 4 shows mass spectrometer measured intensity of various digest products of leucine enkephalin following the described plasma treatment using an AC electrical signal of various different powers;

FIGS. 5A and 5B compare mass spectrometer measured intensities of various digest products of leucine enkephalin following the described plasma treatment using a DC (FIG. 5A) and an AC (FIG. 5B) electrical signal;

FIG. 6 shows mass spectrometer measured intensity of various digest products of methionine enkephalin following the described plasma treatment over a variety of lengths of treatment periods;

FIG. 7 shows mass spectrometer measured intensity of various digest products of creatinine following the described plasma treatment over a variety of lengths of treatment periods;

FIG. 8 shows mass spectrometer measured intensities of various digest products of leucine enkephalin following the described plasma treatment using a DC electrical signal, wherein the leucine enkephalin is treated with DMPO prior to the plasma treatment;

FIG. 9 shows mass spectrometer measured intensities of various digest products of leucine enkephalin following the described plasma treatment using a DC electrical signal, wherein the leucine enkephalin is treated with 4-phosphonoxy-TEMPO prior to the plasma treatment;

FIG. 10 shows mass spectrometer measured intensities of various digest products of leucine enkephalin following the described plasma treatment using a DC electrical signal, wherein the leucine enkephalin is treated with 3-CP prior to the plasma treatment;

FIG. 11 shows mass spectrometer measured intensities of various digest products of leucine enkephalin following the described plasma treatment using a DC electrical signal in the absence of a spin trap/spin scavenger;

FIG. 12 shows DESI-MSI heatmaps of two m/z peaks generated by exposure to positive DC-AP as applied to neat leu-enkephalin, neat bradykinin, and a mixture of the two;

FIG. 13 shows a MALDI-MSI heatmap of a peak at 502.319 m/z in lysates from control cells and cells modified to express the fluorescent protein mNeonGreen, where the lysate is either plasma-naïve or has been exposed to a cold argon plasma setup for one minute; and

FIG. 14 shows a MALDI-MSI heatmap of a peak at 203.172 m/z across tissues which are optionally spiked and/or exposed to a cold argon plasma setup for one minute.

### DETAILED DESCRIPTION OF EMBODIMENTS

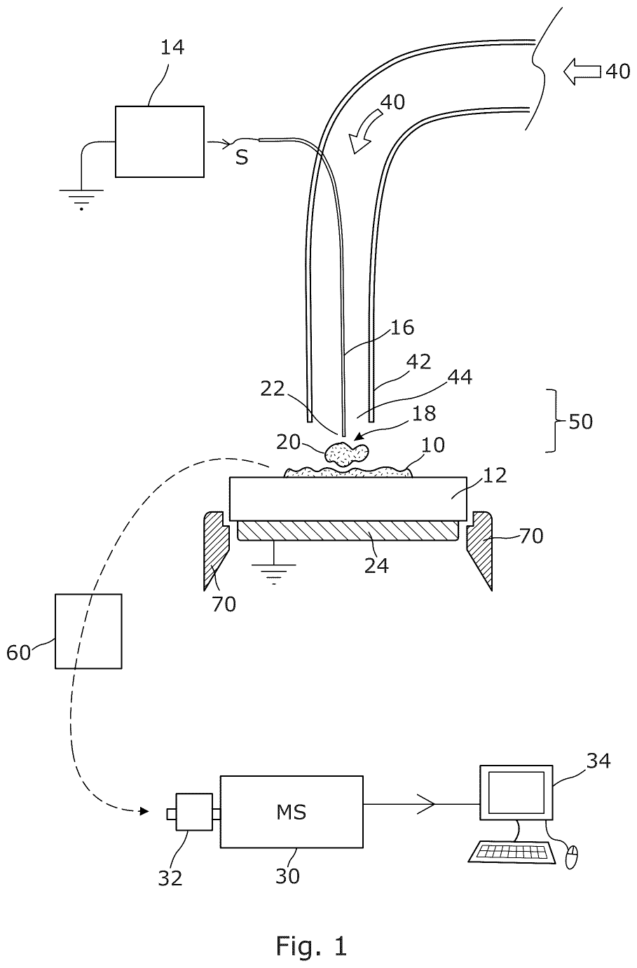

Referring now to FIG. 1 there is shown apparatus for analysing a condensed phase sample, and more particularly organic molecules of a condensed phase sample 10, using a plasma treatment digestion process. The condensed phase sample 10 may be in a solid or a liquid form, or some mixture of the two for example as a gel or some other mixed phase state. In some cases the sample may be a crystalline solid, while in others it may comprise a solution in which the organic molecules are dissolved, a tissue of a human or animal subject such as a biopsy, a cell sample or a blood sample, an environmental sample such as soil or vegetation matter, a food sample, a cell culture (for example of mammalian or plant or insect or microorganism cells) or cell lysate, or engineered or artificial tissues, or any of a wide variety of other condensed phase sample types.

The organic molecules to be analysed may in particular be, or include, proteins and/or peptides, but a wide variety of other organic molecules may similarly be treated and analysed such as amino acids and other small organic molecules, lipids, carbohydrates and other longer chain molecules. Experiments by the inventors which are discussed below demonstrate that the described plasma treatment, when applied to peptides tends to produce c- and z-type ions familiar in mass spectrometry and typical of odd-electron fragmentation, but that the extent of this bond cleavage and consequent fragmentation appears to be dependent on factors such as the length of the plasma treatment.

Where the organic molecules are organic polymer molecules such as proteins and/or peptides, the described techniques are particularly advantageous in that they can be used to fragment the organic molecules along the polymer backbone, for example as shown in the various experimental examples set out below.

In the arrangement of FIG. 1 the condensed phase sample is deposited on, at or in a solid substrate 12 in a consolidated form (i.e. the sample is in the form of a contiguous body of a liquid, solid, or mixture of the two). The substrate may be a microscope slide or a similar substrate, but could be provided with apertures for example being in the form of a mesh so that the gas flow can continue past the sample and carry the plasma into better contact with the sample. The sample is typically also stationary on the substrate. To form a plasma for treating the sample, an electrical signal S is generated by a signal generator 14 and applied to one or more electrodes so as to generate an electrical discharge 18 (which may be described as a corona discharge in at least some embodiments) which in turn generates a plasma 20 proximally to the electrode(s). The condensed phase sample 10 located on the substrate is then exposed to this plasma 20, typically through direct contact between the plasma and the sample, which causes fragmentation of the organic molecules within the condensed phase of the sample itself, for example without desorbing the sample. This process may also be referred to as digestion of the sample.

In some arrangements, for example as illustrated in FIG. 1, the one or more electrodes comprise one or more plasma source electrodes 16 arranged to generate the plasma 20 between the plasma source electrode(s) 16 and the sample 10, and one or more ground plane electrodes 24 or grounding pads disposed on an opposite side of the sample from the plasma source electrode. For example, the ground plane electrode(s) may be separated from the sample by at least a portion of the substrate 12, and may form a part of the substrate.

In other arrangements, both or all of the electrodes may be provided on the same side of the sample as the generated plasma, rather than using one or more ground plane electrodes.

Fragmentation here arises from the breaking of bonds between different substantial parts of the molecule, for example typically C—C or C—N bonds in the case of peptides. Where the molecule is an organic polymer molecule the described techniques are advantageous in particularly causing fragmentation along the backbone of the organic polymer molecule. The term fragmentation as used herein is not meant to cover mere ionisation through loss or gain of one or more electrons or protons. Although some ionisation may indeed occur during this process leading to ionised fragments for example in a liquid solution within the sample, this digestion process typically leaves the sample in the same contiguous and condensed phase form on the substrate as before the plasma treatment started.

The generated plasma used in the described techniques may be described as a cold plasma, a nonthermal plasma, or a low-temperature plasma. Such plasmas can readily be generated by electrical discharge at pressures at or close to atmospheric pressure.

Although this description of the invention does not rely on the plasma fragmentation by the protein being effected by any particular mechanism, the inventors suggest that the energy of the plasma directly cleaves the organic molecule, possibly through a combination of electronic effects such as odd-electron fragmentation, and spatially dependent energetic effects resulting from excited gas particles imparting energy to bonds and atoms.

This plasma treatment of the sample may be carried out for a predetermined time period, typically for a few tens of seconds, but optionally at least 0.1 seconds, at least 1 second, at least 10 seconds, at least 60 seconds, or at least 120 seconds. During this treatment period the sample may be held stationary relative to the plasma and/or the one or more electrodes, or may be moved if required for example to improve effectiveness or consistency of the plasma treatment while remaining consolidated on the substrate. The inventors have found that longer treatment periods result in more fragmentation or digestion, probably including further fragmentation of fragments already produced earlier in the treatment period. Some useful levels of fragmentation were observed after treatment periods of only about 1 to 10 seconds, and shorter treatment periods may also be used, for example of 0.1 seconds or more, if appropriate fragmentation can be achieved in that time.

Desorption of the condensed phase sample for introduction into a mass spectrometer preferably does not take place at the same time as the plasma treatment. Rather, once the plasma treatment has been completed, and the sample is no longer being exposed to the plasma treatment, some or all of the sample comprising the plasma generated fragments of the organic molecules is provided as an input into a mass spectrometer 30, for example via an ionizer 32 of the mass spectrometer. Typically for these purposes, a condensed phase portion of the sample 10 will be removed from the substrate 12 before it is input into the ionizer 32, but optionally the whole substrate could be placed into the ion source of the mass spectrometer for this purpose, depending for example on the design and functionality of the ionizer 32.

The mass spectrometer is then used to detect and/or characterise the fragments of the sample generated by the described plasma treatment. For example, data such as peak intensities for particular mass-to-charge ratios of the fragments may be output by the mass spectrometer 30 to a computer 34 where the data is used to identify, quantify or obtain structural information about one or more of the original organic molecules present in the condensed phase sample, or to characterise the sample in other ways using the mass spectrometer measurements of the generated fragments for example by analysing the data using fingerprinting, statistical, or AI techniques. Note that this detection and/or characterisation of the fragments by the mass spectrometer may include various mass spectrometry techniques which can be used to isolate and further fragment the fragments generated by the plasma treatment, for example to obtain more structural information. Further analytical techniques such as NMR may also be used to characterise the fragments.

In some embodiments, an imaging mass spectrometer may be used to detect or characterise the fragments at multiple locations across the sample, for example in a line, a grid, or some other distribution of locations. This use of an imaging mass spectrometer may be of particular use where the sample as treated by the plasma is a solid sample, and the treated solid sample is then used directly as the sample for the imaging mass spectrometry, so that the structure of the sample remains intact and not delocalised through the period of the plasma treatment and extending until the extraction of material for measurement by the mass spectrometer. If an imaging mass spectrometer is used, this will typically involve the use of a moving stage carrying the substrate past a mass spectrometer input or ionisation point for example as defined by an ionization beam, typically in a raster pattern although other scan patterns may be used. If such a moving stage is used then the described plasma treatment may be carried out with the substrate and sample mounted on the same moving stage before the subsequent imaging mass spectrometry operation.

Note that although FIG. 1 illustrates use of a mass spectrometer to detect and/or characterise the fragments of the sample generated by the described plasma treatment, other suitable analysis methods and corresponding analysis devices or equipment may instead be used for analysing the sample after the plasma digestion process. Such methods and devices include: nuclear magnetic resonance NMR methods and analysers, and various spectroscopic methods and analysers for example operating in the infrared region, in the UV and/or visible regions, using Raman spectroscopy, and using electron paramagnetic resonance EPR, and such other methods and devices may also provide imaging mode analysis of the same similar to the above mentioned imaging mass spectrometer.

Also as illustrated in FIG. 1, a flow of a gas 40 may be provided past the electrical discharge 18 towards or to the sample 10. The gas 40 may assist in enabling the plasma to be initiated and sustained by the electrical discharge, and may also help through the flow of the gas to ensure that the plasma comes into contact with the sample. The gas may be of various types and compositions as discussed further below.

In FIG. 1 a conduit 42 such as a plastic or glass tube has an open end or nozzle 44 facing towards the sample 10, and the electrical discharge takes place proximally to, or within the open end of the conduit, for example by means of the plasma source electrode 16 being implemented as a needle extending along the conduit and terminating in a sharp discharge point 22 close to the open end of the conduit. The plasma source electrode 16 may terminate in this way within or at the end of the conduit 42, or may extend slightly beyond the end of the conduit. In some experiments carried out by the inventors the conduit 42 was provided by a plastic tube of about 10 mm internal diameter, with the plasma source electrode being a stainless steel corona needle passing along the inside of the conduit and protruding from the open end by about 2 mm. In these experiments, the distance from the end of the plasma source electrode to the sample was about 1 mm. Of course, these geometric details such as protrusion distance and distance from the discharge point to the sample may be varied as required.

In other arrangements, rather than using a ground plane electrode 24 disposed on an opposite side of the sample 10 or substrate 12 to the plasma source electrode 16, a ground plane or second electrode could be provided around the tip of the conduit 42, or in other locations on the same side of the sample as the plasma source electrode.

Although the arrangement of FIG. 1 shows a single conduit 42 carrying the flow of gas 40 and with a single plasma source electrode extending along the conduit to generate the plasma proximally to the end of the conduit, a variety of other geometries may be used. For example, electrodes of various other shapes and forms may be used, and do not necessarily extend along a conduit carrying the described flow of gas, but could be located separately to any such conduit. Moreover, multiple such electrodes may be used, or a single such electrode having multiple discharge points 22 for example in the form of a comb or brush. Similarly, multiple conduits delivering the described flow of gas may be used to help initiate and sustain the plasma and to direct the plasma to the condensed phase sample.

More generally, at least the one or more plasma source electrodes 16 including the discharge points 22, and optionally also components such as the conduit 42, may be described as a plasma generator 50, to which the signal generator 14 provides the electrical signal S in order to generate the plasma. In some embodiments, particular apparatus may also be provided to carry the, or portions of, the sample after the plasma treatment to the mass spectrometer 30 and/or ionizer 32, and in FIG. 1 this is illustrated generically as sample conveyor mechanism 60. This could take the form of some robotic or other mechanical equipment, could involve liquid transfer for example using pipetting as described above, or other liquid transfer means, or transfer of solids material from the sample or the whole substrate, and in particular could be an automatic mechanism so that transfer of plasma treated sample from the plasma treatment to the mass spectrometer may be carried out automatically and without human intervention. As shown in FIG. 1, the apparatus may also comprise a substrate carrier 70 for receiving the substrate and/or sample in a suitable or correct position for plasma treatment by the plasma generator 50. Such a substrate carrier 70 may for example comprise a suitably sized and shaped frame for accepting the substrate 12.

Various different forms of electrical signal S and related circuit arrangements, including arrangements of the substrate carrying the sample, may be used in order to generate the plasma. For example, in some arrangements the signal S may be a DC signal, and optionally a DC signal of substantially constant voltage, or of varying voltage, during the treatment period. For example, the DC signal may have a voltage of between about 1000 and 10,000 V. Such a DC signal may also have a duty cycle within which some of the time (for example 50% of the treatment period) the voltage is small or zero, and at other times (for example the other 50% of the treatment period) it is at a higher level so as to generate the plasma. If a DC form of signal S is used then a conductive return current path from the sample will typically be required, for example by using for the substrate 12 a microscope slide or other insulating component provided with a conductive plane or grid to provide a local ground plane electrode in contact with the sample and a return current path for the signal S from that ground plane electrode (not shown in FIG. 1), or by using a conductive substrate connected in a similar manner. In some arrangements this may conveniently be implemented by using as substrate 12 a whole or a fragment of a commercially available microscope slide which is already provided with an electrode in the form of a thin conductive layer such as indium tin oxide on the upper surface of the substrate which is to carry the sample.

The inventors have found that although a DC signal S can be used to form a plasma effective to fragment the organic molecules of the sample for subsequent mass spectroscopy and detection of the fragments, it can be challenging to generate a suitable plasma except at relatively low effective electrical powers for example of the order of around one Watt or less. In other arrangements, therefore, the signal S may be an AC signal, or more particularly a radiofrequency signal. For example, the AC signal may have a peak to peak voltage of between about 1000 and 10,000 V, and more particularly between about 2000 and 5000 V. The AC signal may typically be substantially sinusoidal, or at least have a main or dominant frequency mode. The AC signal may then have a frequency (or frequency of the main or dominant mode) for example from about 10 kHz to 10 MHz, or more particularly from about 100 kHz to 1 MHz, or 100 kHz to 5 MHz, or more generally may be a radio frequency AC signal. The electrical power of the AC signal may then be of the order of a few tens of Watts, for example in the range from 1 to 1000 Watts or more particularly from 10 to 300 Watts. The radiofrequency signal could be a digital radiofrequency signal.

If an AC form of signal S is used with a suitable AC frequency and electrical arrangements then it may not be necessary to provide any conductive return current path directly from the sample. For example, instead of locating the sample on an electrically conductive surface or other electrode of the substrate 12, the ground plane electrode 24 may be provided as a grounding pad, for example being located on a lower surface an insulating substrate 12 or elsewhere not in direct contact with the sample. Such a grounding pad 24 typically provides an electrical ground connection in common with the signal generator 14, or a more direct or deliberate electrical connection between the two could be used.

In some particular experiments carried out by the inventors, a radio frequency AC signal S was provided by using a ForceTriad® electrosurgical signal generator manufactured by Medtronic Inc. This was used to generate a radiofrequency AC signal comprising damped sinusoidal bursts having an AC frequency of about 472 kHz. These damped sinusoidal bursts were provided with a randomised repetition centred at about 21.7 kHz, with a 4.6% overall duty cycle. The average power over the treatment period was between about 20 W and 120 W, and the peak to peak voltage of the signal S was around 2000-3700V.

As noted above, the arrangement of FIG. 1 provides for the presence at, or flow past the electrical discharge 18 and/or discharge point 22, of a particular gas 40. The use of suitable gas species within this gas can permit or assist in the initiation and sustaining of the plasma, and a flow of the gas may also assist in directing the plasma onto and into contact with the sample. The inventors have found that a suitable species for the flow of gas is argon, but that other noble gases such as helium, and other relatively unreactive gases such as nitrogen may also be used, as may mixtures of such gases. For example, the gas may comprise at least 50%, or at least 90%, of one or more of: a noble gas, argon, helium, neon, and nitrogen. Argon was found by the inventors to be particularly effective in experiments in supporting fragmentation of organic molecules in the sample for mass spectrometry, and this might in part be due to the low breakdown voltage of argon relative to air. The gas 40 may also contain one or more dopants, such as fluoranthene, at much lower levels of concentration to support the above and other functions.

The volumetric flow rate of the gas, or velocity of the gas for example at the outlet or nozzle 44, may be adjusted according to need and other aspects of the equipment being used such as size of the nozzle 44, electrical power of the signal S, and distances between and geometries of the nozzle 44, the discharge point 22, and the sample 10. However, typically a pressure of a source of the flow gas of about 10 kPa (0.1 bar) above the ambient pressure at the plasma may be used, or a flow rate of the order of 0.1-1.0 litres/minute.

In the arrangements similar to that of FIG. 1, the gas may be directed along the conduit 42 to the electrical discharge 18 and/or discharge point 22, but such flow or location of the gas at these points can be achieved in other ways, for example by locating the sample and electrode within a closed container flooded with the gas 40, by using one or more conduits or nozzles to deliver the gas from other directions for example laterally across the sample, and so forth. However, there may be advantages in ensuring that the flow of gas is directed in a direction which is from the electrical discharge 18 directly towards the sample so that the plasma is urged by the gas flow into contact with the sample.

An advantage of the described arrangements for generating fragments of organic molecules for mass spectrometry is that they can be carried out at ambient pressures, thereby avoiding need for any vacuum or pressure vessel. However, the described arrangements may more generally be implemented at partial vacuums or elevated pressures if desired, by way of example in a range from about 30 to 300 kPa.

The condensed phase sample 10 can be prepared for the described plasma treatment in various ways. For example, in the experiments described below a solid sample of an acetate salt of an organic molecule such as a peptide was dissolved in a 30:70 mixture of water and methanol, then pipetted on to a suitable microscope slide substrate 12 and allowed to dry before plasma treatment. However, a wide variety of other sample preparation methods may be used, including methods with minimal or no processing. For example, biopsy or sample cell cultures, blood or other tissue samples, environmental samples, food samples and so forth may all be applied to a suitable substrate for carrying out the plasma treatment.

However, in some examples, a condensed phase sample may be subject to the described plasma treatment without applying it to a particular substrate, for example by carrying out the treatment in situ externally or internally, for example during surgery on a human or animal body. For example, the plasma treatment could be carried out during surgery using an electrosurgery instrument which generates a plasma for surgical purposes but which also carries out the described fragmentation, with the fragments then being extracted from the surgery site either automatically or manually for mass spectrometry.

The inventors have also found that addition of certain reagents or substances to the sample can be used to enhance the production generally or production of particular fragments, and/or to enhance the visibility of such fragments in the data output by the mass spectrometer. For example, the sample may be treated with a compound selected from a spin trap and a spin scavenger. Such compounds may be useful where the fragments of the organic molecules resulting from the plasma treatment include short-lived radicals (species with at least one unpaired valence electron) that would otherwise have decomposed prior to their detection in the mass spectrometer. In particular, the spin trap or spin scavenger may react with these short-lived radicals to form stabilised species that can then be detected. By “spin trap” it is meant a diamagnetic compound that is capable of reacting with an unstable (short-lived) radical to form a stabilised addition product in the form of a further radical. By “spin scavenger” it is meant a radical that is capable of reacting with an unstable (short-lived) radical to form a stabilised addition product in the form of a diamagnetic (i.e. non-radical) compound. These terms are well known to those skilled in the detection of radicals. “Spin scavengers” are also known as “radical scavengers”.

While the concept of using radical addition reactions to help detect short-lived radicals is known for example in analytical techniques such as electron paramagnetic resonance (EPR) spectroscopy, mass spectrometry detects mass changes rather than unpaired spins. In particular, the inventors propose to use spin traps and spin scavengers to treat a condensed phase sample before it enters a mass spectrometer. One advantage of this approach is that the spin trap or spin scavenger is already present when the fragments are generated, such that they can be trapped or scavenged essentially instantaneously. In contrast, in most existing uses of spin traps and spin scavengers in analytical methods (e.g. EPR spectroscopy), the compound is added after the radicals have already been generated, meaning that there is a risk of particularly short-lived radicals decomposing before they have had chance to react with the compound.

The inventors have found that certain plasma treatments are more likely to generate radicals and are therefore more likely to benefit from fragment stabilisation using a spin trap or spin scavenger. For instance, when a DC signal is used to generate the plasma under the conditions described herein, at least some of the fragments generated from the organic molecules may be in the form of radicals. In contrast, under the conditions the inventors have employed to date, they have not observed products formed by the reaction of a peptide with spin traps or spin scavengers after exposure to a treatment plasma generated using an AC electrical signal. Accordingly, the treatment of the condensed phase sample with a compound selected from a spin trap and a spin scavenger may be particularly advantageous when a DC signal is used.

Spin traps and spin scavengers are known in the art, and the nature of the spin trap or spin scavenger that may be used in the present invention is not particularly limited, provided it is able to react with the radicals generated from a given sample to form a stabilised adduct that can then be detected. Where the compound is a spin trap, it may, for instance, be a nitrone or a nitroso compound. By “nitrone” it is meant an N-oxide of an imine. Any nitrone spin traps, either in cyclic or acyclic configuration may be used in the present invention. Suitable cyclic nitrones include 5- or 6-membered ring structures in which the nitrogen atom of the N-oxide forms part of the ring. In some embodiments, the nitrone is a pyrroline N-oxide, wherein the pyrroline ring is optionally substituted with one or more alkyl groups, more preferably a 5,5-dialkyl-1-pyrroline-N-oxide. Each alkyl group is independently selected from C₁-C₄ alkyl, for instance. Still more preferably, the nitrone is 5,5-dimethyl-1-pyrroline-N-oxide (DMPO). In other embodiments, the N-oxide group does not form part of a ring structure. For instance, the nitrone may be an alpha-aryl N—C₁₋₄alkyl nitrone, preferably an alpha-phenyl-N-butyl nitrone, still more preferably alpha-phenyl N-tertiary-butyl nitrone (PBN). Other suitable nitrone spin traps include alpha-pyridine-N′-oxide-N-tertiary-butylnitrone (POBN), 3,3,5,5-tetramethyl-1-pyrroline-N-oxide (M₄PO) and 5-(diethoxyphosphoryl)-5-methyl-1-pyrroline-N-oxide (DEPMPO). Similarly, any suitable nitroso compound spin traps may be used in the present invention. By “nitroso compound” it is meant a nitric oxide (—N═O) group attached to an organic moiety. Suitable nitroso compounds include dihalo-nitroso-benzenesulfonic acids, wherein halo is Cl, Br or I, preferably Br. For instance, the nitroso compound may be a 3,5-dihalo-4-nitrosobenzenesulfonic acid, more preferably 3,5-dibromo-4-nitrobenzenesulfonic acid (DBNBS). Alternatively, the nitroso compound may be a 2-C₁₋₄alkyl-2-nitrosopropane, more preferably 2-methyl-2-nitrosopropane (MNP).

Where the compound is a spin scavenger, it may, for instance, be an aminoxyl radical. Any aminoxyl spin scavengers, either in cyclic or acyclic configuration may be used in the present invention. Suitable cyclic aminoxyl radicals include 5- or 6-membered ring structures in which the nitrogen atom of the aminoxyl group forms part of the ring. In some embodiments, the cyclic aminoxyl radical is a piperidinyloxy radical, wherein the piperidinyl ring is optionally substituted with one or more alkyl groups and optionally one or more phosphonoxy groups. For instance, the cyclic aminoxyl radical may be a 2,2,6,6-tetraalkyl-1-piperidinyloxy or a 4-phosphonoxy-2,2,6,6-tetraalkyl-1-piperidinyloxy radical, wherein each alkyl group is independently selected from C₁-C₄ alkyl, for instance. In these embodiments, the cyclic aminoxyl radical is preferably (2,2,6,6-tetramethylpiperidin-1-yl)oxyl (TEMPO) or 4-phosphonooxy-2,2,6,6-tetramethyl-1-piperidinyloxy 4-phosphonoxy-TEMPO (4-phosphonoxy-TEMPO). In other embodiments, the cyclic aminoxyl radical is a pyrrolidinyloxy radical, wherein the pyrrolidinyl ring is optionally substituted with one or more alkyl groups and optionally one or more cyano groups. For instance, the cyclic aminoxyl radical may be a 3-cyano-2,2,6,6-tetraalkyl-1-pyrrolidinyloxy radical, wherein each alkyl group is independently selected from C₁-C₄ alkyl, for instance. In these embodiments, the cyclic aminoxyl radical is preferably 3-cyano-2,2,5,5-tetramethyl-1-pyrrolidinyloxy (3-cyano-proxyl, or 3-CP).

The manner in which the condensed phase sample is treated with the compound is not particularly limited, provided the compound remains in contact with the sample when the sample is exposed to the plasma. For instance, the compound may be provided in the form of solution in water and/or a non-aqueous solvent, wherein the solution is applied to the condensed phase sample (e.g. by pipetting the solution onto the sample). For instance, in the experiments below the spin trap or spin scavenger was dissolved in a 30:70 mixture of water and methanol, then pipetted onto the solid peptide sample on the microscopic slide substrate 12 and allowed to dry. Alternatively, the compound may be applied to the condensed phase sample as a solid. In these embodiments, the compound and the condensed phase sample are preferably combined to form a homogenous mixture. In other embodiments, a solution of the compound is combined with a solution of the condensed phase sample to form a mixture and the mixture then allowed to dry. Other suitable means for treating the sample with the compound will be apparent to those skilled in the art. In the treatment step, the compound and the organic molecules may be present in a weight ratio of around 1:1, for example in a range from 0.1:1 to 10:1.

Following any required preparation steps as discussed above, the condensed phase sample 10 is then treated as already described with the plasma so as to generate fragments of organic molecules within the condensed phase sample. Such treatment may be applied to the sample for a particular length of time found suitable by experimentation for generating those molecule fragments which are of particular utility or interest in characterising the molecules or sample at suitable levels of concentration. Other parameters which may be optimized through experience or suitable experimentation to generate molecule fragments of particular utility at suitable concentrations may include the voltage, power, current, and/or AC frequency of the electrical signal S as already discussed above, the constitution and flow rate of the gas 40, structuring or surface preparation of the sample on the substrate, and so forth.

Once treatment of the condensed phase sample by the plasma 20 has been completed, a portion of the sample is transferred to a mass spectrometer for analysis, for example as also illustrated in FIG. 1. For example, if the sample is a crystallised solid arising from evaporation of a solution as mentioned above, then following plasma treatment a small amount of solvent may again be pipetted onto the sample so that it dissolves, and the resulting solution transferred to the ionizer 32 or other input element of the mass spectrometer. In other examples, material of the sample may already be in liquid form and may be transferred to the mass spectrometer as such, or solid material from the sample may be transferred while still solid. In some examples, some part or all of the substrate carrying the treated sample may be transferred as a whole and placed in the ioniser 32, or otherwise used for injection of sample material into the mass spectrometer. Advantageously, the mass spectrometer may be an imaging mass spectrometer so as to take particular advantage of the described plasma treatment which be used while keeping the sample structurally intact during treatment, especially if the sample is in a solid form.

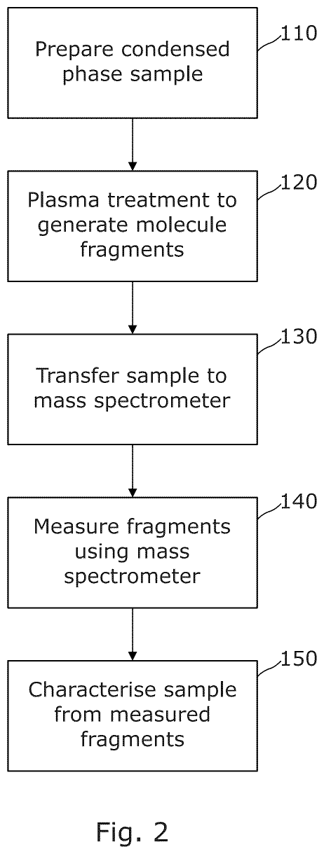

The described methods according to the invention are summarised in FIG. 2 in which a flow chart of steps for analysing organic molecules of a condensed phase sample are described. In step 110 the condensed phase sample is prepared. As discussed above, this preparation could be minimal for example in applying a sample to a suitable substrate, or a sample already in situ for example on or in a human or animal subject could be used. Sample preparation may involve addition of further reagents or substances such as spin trap or spin scavenger materials as discussed above.

In step 120 the described plasma treatment is carried out, typically for a predetermined treatment period, and using plasma generation parameters such as electrical signal power and frequency, and other parameters such as gas type and flow rate, found suitable for the required level and character of fragmentation of organic molecules within the sample.

In step 130 some or all of the plasma treated sample is transferred to a mass spectrometer for detection of fragments of the sample organic molecules generated by the plasma treatment. This step may involve the measurement of particular known and expected fragments, or more generally obtaining of a mass spectrum or other data for further analysis.

Finally, at step 140 the original sample is characterised in some way using the data from the mass spectrometer, for example by determining the presence, absence or concentration of particular molecules in the original sample, by analysing the data using fingerprinting, statistical, or AI techniques, or in other ways, at least in part by using data in the mass spectrum relating to the fragments generated using the plasma treatment. In some instances, the mass spectrum may be used as data to help in the identification or characterisation of unknown or uncharacterised molecules in the sample.

A number of different experiments carried out by the inventors to demonstrate efficacy and implementation of the invention will now be described. At least some of these examples relate to the fragmentation of organic polymer molecules along the polymer backbone, with subsequent detection of the backbone fragmented components.

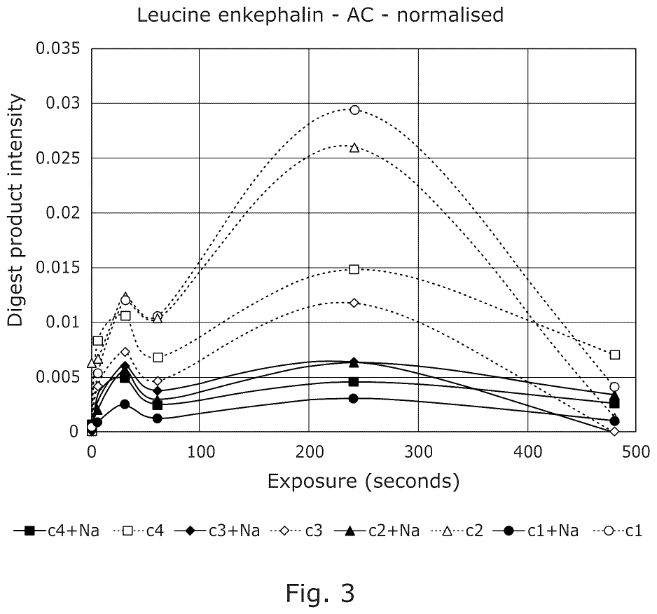

FIG. 3 illustrates results of carrying out the described plasma digestion and subsequent mass spectrometry steps on a sample of leucine enkephalin using a plasma generated using a radiofrequency AC signal S within an argon gas flow. The AC signal was generated using the ForceTriad® signal generator mentioned above, operating at a 35 Watt output, with the AC frequency at 4723 kHz and the duty cycle as already described. The experimental arrangement was also as already detailed above and shown in FIG. 1, using for electrode 16 a stainless steel corona discharge needle extending about 2 mm from the open end of a plastic tube acting as conduit 42 with internal diameter of 10 mm, with an argon gas flow at a pressure of 10 kPa above ambient pressure supplied so as to flow along the tube past the end of the needle and to the sample. The experiment was otherwise carried out in ambient conditions without any particular arrangements being made for separation from the laboratory ambient air.

The experiment was repeated 6 times, each time on a fresh untreated sample and with a different plasma treatment period, of: 0, 5, 30, 60, 240 and 480 seconds. Each time after treatment a solvent was pipetted onto the sample to dissolve some of the treated leucine enkephalin, then some of that solute was injected into a Bruker TIMS-TOF mass spectrometer with a mass/charge accuracy of about +/−0.005 Da.

FIG. 3 then shows the digest product intensity output by the mass spectrometer for each of c1 to c4 fragments of the leucine enkephalin after the stated treatment time, and also the digest product intensity for corresponding Na adduct ions. As is conventional in mass spectrometry, the c1 fragment is the product generated by cleavage of the first N—C bond in the peptide chain from the N-terminus (not including the N—C bond of the primary amine N-terminus itself), with c2-c4 fragments arising from cleavage of the next three C—N bonds in the peptide chain. In FIG. 3 these digest product intensities are shown normalised by the M+H ion intensity measured by the mass spectrometer at the same time (i.e. the parent ion corresponding to the whole, unfragmented molecule). It can be seen that significant digestion into all shown fragment species has taken place after just 5 seconds of treatment, although normalised product intensity is greatest following 240 seconds of treatment in this case, and diminishes again after that, possibly due to further fragmentation of the measured products into further, possibly smaller, fragmentation products.

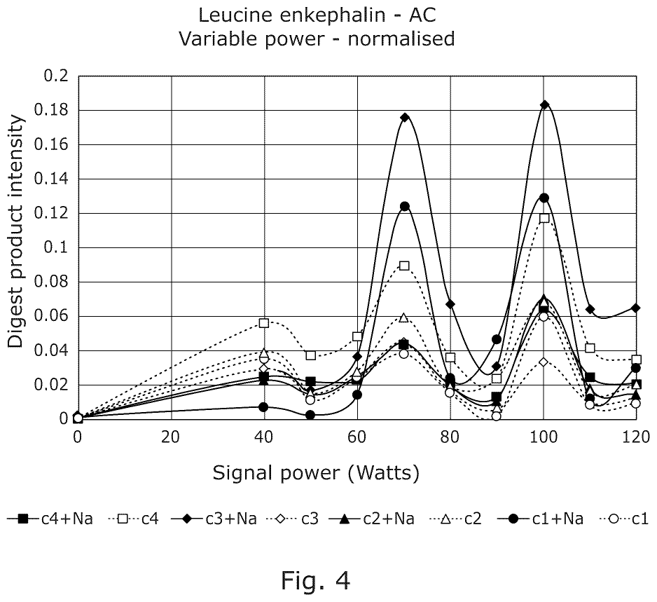

FIG. 4 shows results of the same experimental set up and conditions, again applied to leucine enkephalin samples, but this time using a 60 second treatment period for all samples, with each sample being treated using a plasma generated operating the Force Triad® signal generator at a different power level. Again, the intensities of the digest products measure by the Bruker mass spectrometer are shown normalised against the M+H ion measure. A clear increase in digest product intensity with increasing signal power is evident, although significant peaks also occur at 70 and 100 Watts, which may be due to particular features of the operation of the signal generator.

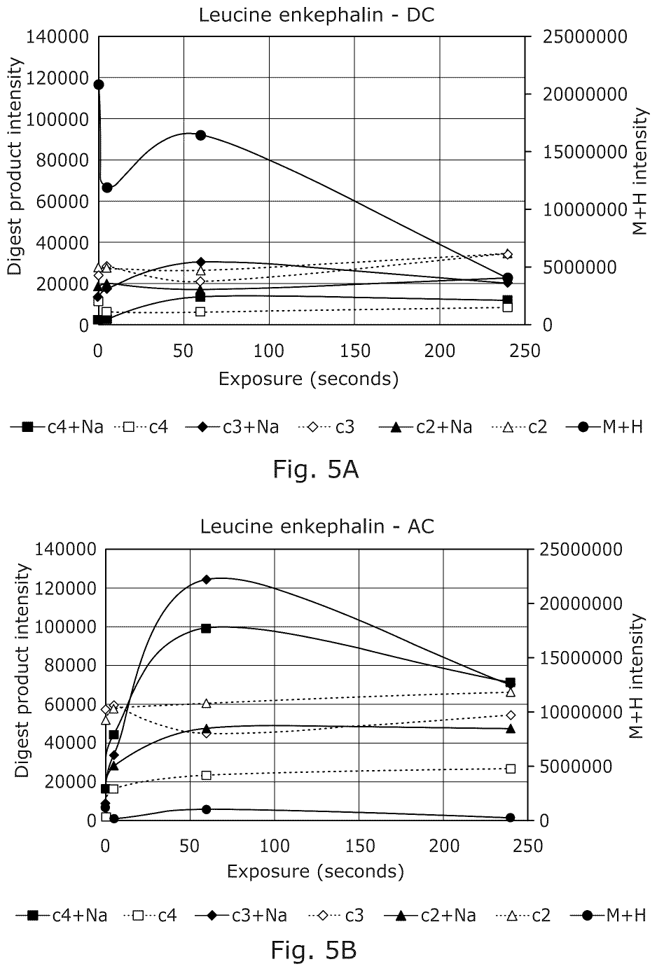

FIGS. 5A and 5B show corresponding experiments on leucine enkephalin samples, but in which a DC signal (FIG. 5A) and an AC signal (FIG. 5B) were used respectively to drive the electrode 16 to generate the plasma. For FIG. 5A, a constant DC signal was experimentally adjusted to develop a visually optimal plasma 20 in terms of intensity and extent, which was achieved using a 5 kV voltage and a current of about 0.1 mA. For FIG. 5A the above ForceTriad® signal generator was used with a 120 Watt setting for the output AC signal again to achieve a visually optimal plasma 20 in terms of intensity and extent, which was of considerably stronger intensity than could be achieved using the DC signal. Samples were subject to plasma treatment using treatment times of 5 seconds, 60 seconds and 240 seconds.

For both these graphs the digest product intensities are plotted on the same (left axis) scales, but are not normalised to the M+H parent ion which is plotted on a different (right axis) scale. Clearly, when using the AC signal much higher digest product intensities are obtained, as well as a lower parent ion intensity, making the signal to noise ratio of the normalised data far superior. Note also that although the intensities of the different fragment species are broadly in the same order or intensity for both DC and AC there are some significant differences, so either signal type might be used depending on particular fragments that are to be measured. In particular, the ratios of fragment to parent ion intensities are much greater in the case of the AC signal, indicating a much higher efficiency of the AC method.

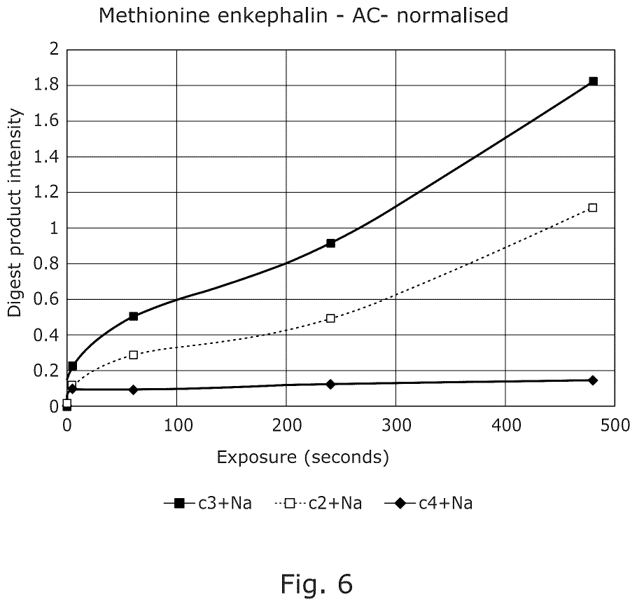

FIG. 6 is similar to FIG. 3, but with plasma digestion treatment and subsequent mass spectrometry applied to a sample of a slightly heavier peptide, methionine enkephalin, with treatment times of 5, 60, 240 and 480 seconds. As for FIGS. 3 and 4, the data is normalised to the intensity of the M+H parent ion, and in this case only Na adduct ions of the c2, c3 and c4 ions are shown, with a strong increase in intensity of the c2 and c3 ions over time, but the c4 ion intensity not rising very much after the first 5 seconds.

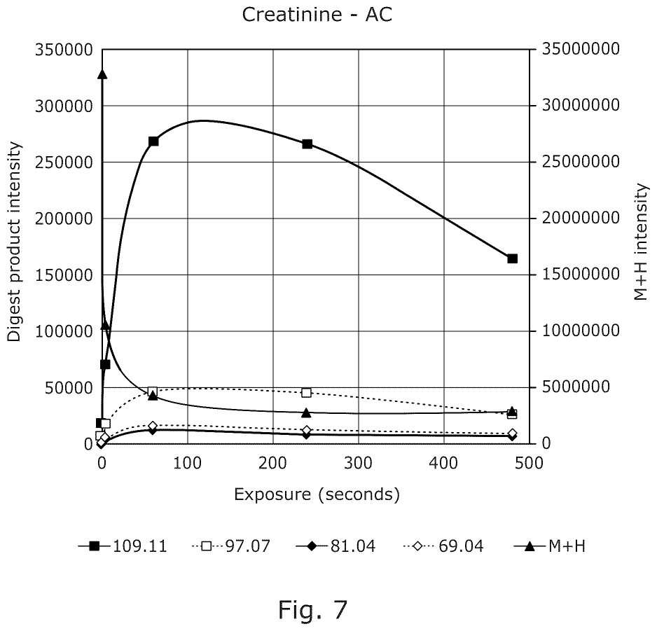

FIG. 7 then demonstrates the described plasma digestion and mass spectrometry technique applied to a creatinine sample prepared in the same way as the peptide samples used above. Creatinine is not a peptide, and has a molecular weight of rather less than the peptides used above, but the plasma digestion still leads to useful fragmentations over all treatment periods of 5, 60, 240 and 480 seconds. The digest product intensities (left scale) are not normalised in this figure, so the intensity of the M+H parent ion (right scale) is also shown. Tentatively the plotted fragments are identified by mass/charge ratios as the Na adducts of 109.11=C₄H₁₂N₂, 97.07=C₃H₁₀N₂, 81.04=C₂H₆N₂ and 69.04=CH₆N₂.

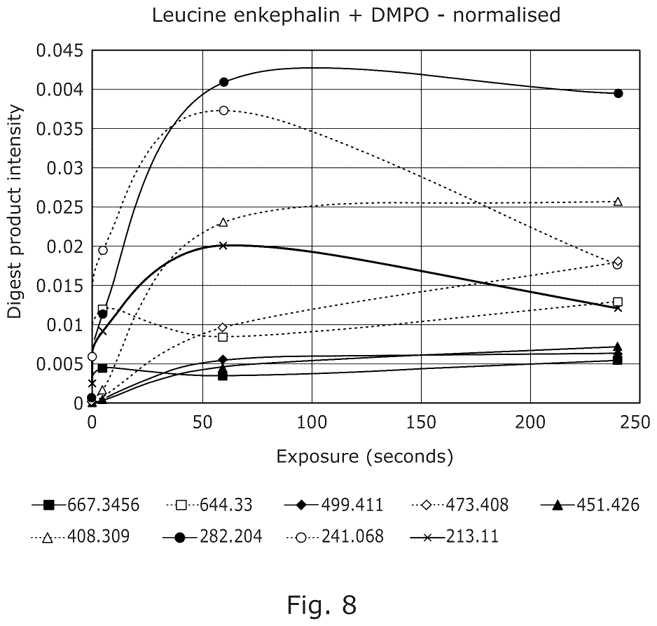

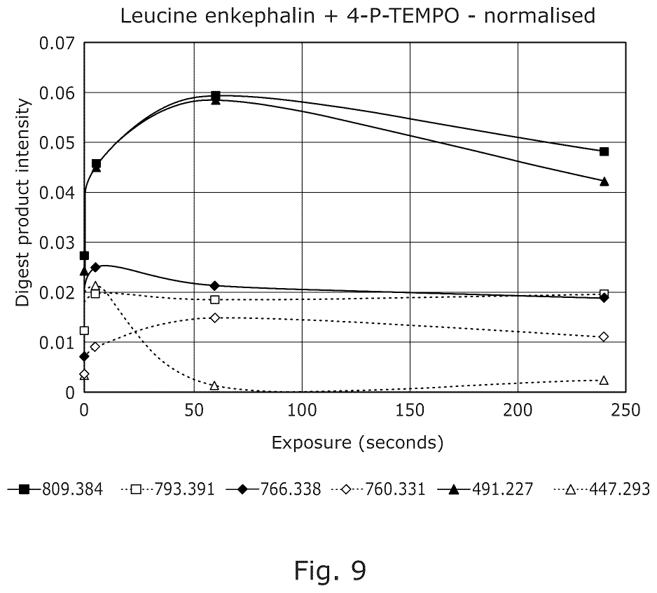

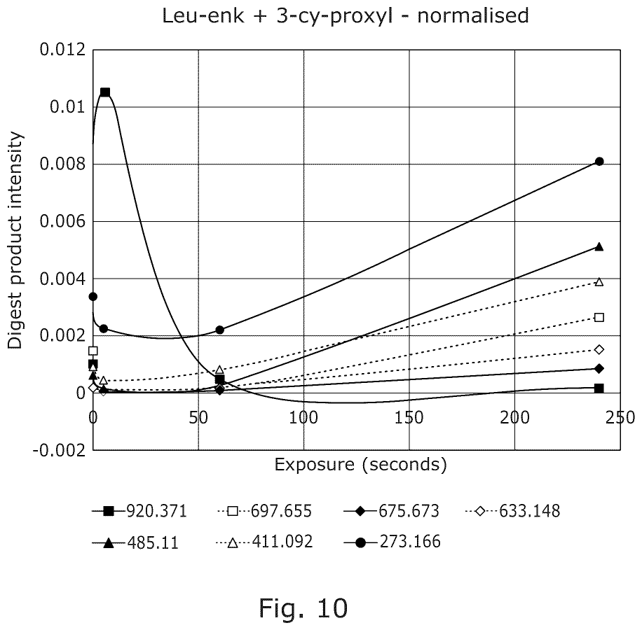

FIGS. 8 to 10 then demonstrate the described plasma digestion and mass spectrometry technique applied to leucine enkephalin samples treated with different spin traps and spin scavengers. In each figure, the ordinate axis represents digest product intensity for each fragment type as output by the mass spectrometer (in each case normalised to the intensity of the M+H parent ion), and identified fragments are labelled using their mass/charge ratio.

In particular, for the experiment of FIG. 8, DMPO (a spin trap) was used, while in the experiments of FIGS. 9 and 10, 4-phosphonoxy-TEMPO and 3-cy-proxyl (both spin scavengers) were used respectively. In each experiment, 2 μL of the spin trap/spin scavenger at a concentration of 1 mg/mL in a 30:70 mixture of water and methanol were pipetted onto the solid leucine enkephalin on the slide. The treated sample was then allowed to dry before plasma generation using a DC signal with the same plasma generation and treatment conditions as for FIG. 5A, and mass spectrometry analysis of the resulting fragments using a Bruker TIMS-ToF mass spectrometer with a mass/charge accuracy of about +/−0.005.

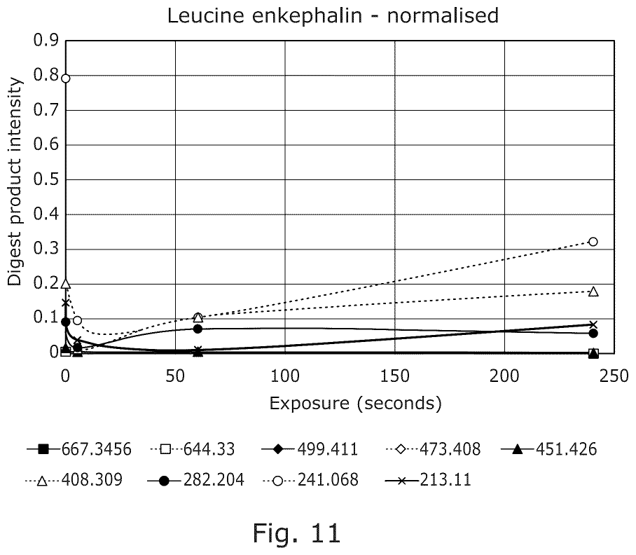

FIG. 11 then shows the results of the same experiment but without the application of any spin trap/spin scavenger to the leucine enkephalin sample. The data is again normalised to the intensity of the M+H parent ion. As can be seen from FIGS. 8 to 11, the spin traps/spin scavengers all enabled significant quantities of various fragments to be detected following 60 seconds of plasma exposure, whereas no fragments were detected at significant levels for this exposure period in the absence of a spin trap/spin scavenger. The observed fragments are yet to be assigned, other than the addition peak for the leucine enkephalin-spin trap/spin scavenger adduct in each case (667.3456 for leucine enkephalin+DMPO, 809.384 for leucine enkephalin+4-phosphonoxy-TEMPO, and 697.655 for leucine enkephalin+3-cy-proxyl).

To further demonstrate the invention, a direct current-Argon Plasma (DC-AP) discharge source consisting of a 5 kV HVDC power supply (Stanford Research Systems PS350) was attached to a 1 MΩ resistor and a brass t-piece (Swagelok) containing a corona needle within an isolating plastic sheath, and gas tubing. Well-characterised peptide standards such as leu-enkephalin and bradykinin (Merck, 1 mg/mL) were used as standards to investigate the effect of the plasma; consistent volumes (2.5 μL) were spotted onto indium-tin oxide slides (Merck, 70-100 Ω/sq), allowed to dry, and exposed to the positive or negative corona-induced plasma at a distance of 12 mm from the corona tip to ITO slide for between 5 seconds and 4 minutes. Activating the DC-AP source under a gas flow of 0.5 bar of argon resulted in a purple glow discharge at the tip of the corona pin.

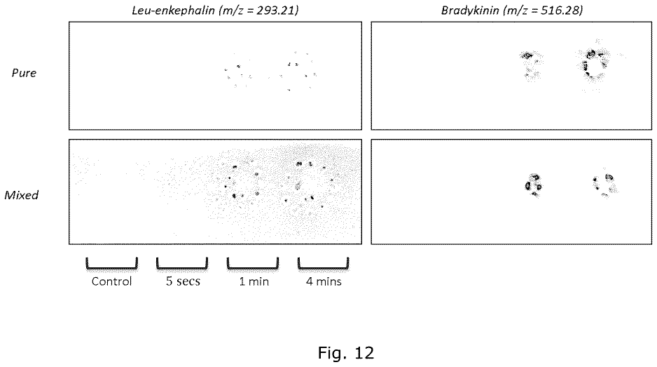

The DC plasma-treated substrates were then either directly analysed by DESI imaging or dissolved in 60 μL 30:70 water:methanol and analysed by direct infusion ESI-MS or LC-MS. Exposure of the standards to the DC argon plasma generated, in subsequent DESI-MS analysis, mass peaks which were consistent, unique, and spatially resolved. An ITO slide was divided into four segments and standards were spotted onto each area—these segments were exposed for 0, 5, 60, or 240 seconds to the plasma before DESI analysis. In this particular case, the peaks generated were not easily elucidated by MS/MS, but clearly demonstrated the utility of plasma-mediated reactions with peptides—it also demonstrated that increasing plasma exposure also increased peak intensity. See the resulting FIG. 12, in which the peak at 293.21 m/z is only present when leu-enkephalin is exposed to DC-AP, while the peak at 516.28 is only present when bradykinin is exposed to DC-AP. Both increase in intensity with increased exposure to the plasma.

To understand the fragmentation mechanism and to elucidate the structures of identified peaks, 5,5-dimethyl-1-pyrroline N-oxide (DMPO) was added to the standards before exposure to the plasma. DMPO is a popular spin-trapping compound, typically utilised in Electron Paramagnetic Resonance (EPR) spectroscopy, which reacts selectively with molecules with unpaired electrons-most notably radical compounds such as those being generated with exposure to the DC argon plasma. The addition of an equal wt/wt volume of DMPO to the standards before exposure resulted in the generation of spin-trapped radicals, easily detectable by LC-MS.

Analysis of the data demonstrated the presence of many peaks absent in the control sample but present in the exposed samples. The major peak at 667.35 m/z, generated by application of the plasma source to a leu-enkephalin-DMPO standard, was hypothesised to be an addition of the two compounds, joined at the phenolic hydroxyl group of the peptide via the Forrester-Hepburn mechanism. An additional major peak expressed at 8.50-8.60 mins in the chromatogram, at 263.14 m/z, was also resolved; this was annotated as the product of coupling between the leu-enkephalin a1 ion and the DMPO, followed by an oxidation.

The use of the DC-AP source on solid-phase samples described produces fragments which are spatially resolvable, consistent, and unique to the exposed peptide. Should a spin-trap compound like DMPO be added before exposure, the DMPO couples to the peptide to produce easily predictable fragments; this reaction also produces several side-products from either the breakdown of the DMPO-peptide conjugate, or the reaction of DMPO with the breakdown products of the peptide.

Use of a radiofrequency (RF) current to generate the plasma can be used to achieve sample digestion without the use of expensive electrically conductive ITO slides, In further demonstrations of the invention, cold RF argon plasmas were generated using an electrosurgical handpiece (Erbe) modified by replacement of the knife blade with a corona pin. A short length of polypropylene tubing with internal diameter of about 10 mm was used to direct a flow of argon gas from the tip, with the tip itself slightly protruding. The handpiece was connected to the unipolar socket of a radiofrequency alternating current generator. Glass slides with dry standards spotted onto were placed on top of a dispersive grounding pad connected to the generator. The activation of the device with 0.1 bar argon flowing generated a purple plasma, which covered a roughly 30 mm diameter area directly below the corona tip. After exposure, the standards were dissolved in 60 μL 50% acetonitrile and analysed by direct infusion.

The digestion products generated included ‘classical’ abc-xyz peptide fragments (particularly c- and z-type fragments), as well as neutral loss products such as [M-COOH+H]+. The presence of c- and z-type fragments suggests a mechanism more heavily reliant on free electron- or radical-mediated reactions than the DC setup, as in-spec fragmentation methods such as Electron Transfer Dissociation (ETD) and Electron Capture Dissociation (ECD) often produce these fragments through charge remote fragmentation characteristic of odd-electron reactions. Increased exposure to the RF-AP tended to reduce the intensity of higher (>500) m/z peaks, while increasing the intensity of lower (<150) m/z peaks (table 1)—for example, increased exposure time leads to a lower intensity of the peak at 464.19 (annotated as [c4+Na]+), but a higher intensity of the peak at 120.08 (annotated as [Phe-COOH—H2+H]+).

Several notable peaks and their respective annotations are enumerated in Table 1 below. The peaks generated included c-type fragments, as well as derivatives of a-, c-, z-, and y-type fragments, in addition to neutral losses from the parent ion.

TABLE 1
Notable fragments, their annotations, and their
absolute intensities from direct infusion analysis
of Leu-enkephalin exposed to the RF-AP source.

| Peak | Annotation | Control | 5 seconds | 30 secs | 1 minute |
|---|---|---|---|---|---|
| 556.28 | M + H | 2.9e5 | 5.3e5 | 2.7e5 | 5.4e4 |
| 550.26 | M—CO + Na | — | 5.3e2 | 5.6e2 | — |
| 534.27 | M—COOH + Na | — | 6.7e2 | 1.2e3 | 1.4e2 |
| 532.25 | M—COOH—H₂ + | — | 1.9e2 | 2.7e2 | — |
| | Na | | | | |
| 528.28 | M—CO + H | — | 1.8e2 | 1.5e2 | 3.7e1 |
| 512.29 | M—COOH + H | — | 3.0e3 | 3.0e3 | 9.1e2 |
| 510.27 | M—COOH—H₂ + | — | 2.2e3 | 1.7e3 | 4.8e2 |
| | H | | | | |
| 464.19 | c₄-H + Na | — | 2.7e2 | 3.8e2 | 2.4e1 |
| 442.21 | c₄ | 1.9e1 | 1.5e3 | 1.3e3 | 1.6e2 |
| 400.18 | c₄-COOH + H | 1.2e1 | 5.1e2 | 6.3e2 | 3.2e1 |
| 356.19 | z₄-COOH + Na | — | 1.9e3 | 4.3e3 | — |
| 332.20 | z₄-COOH + H | — | 2.0e2 | 2.3e2 | — |
| 299.17 | z₃-COOH + Na | — | 3.2e3 | 8.0e3 | — |
| 295.14 | c₃ | — | 1.3e3 | 1.2e3 | 8.4e1 |
| 277.19 | z₃-COOH + H | — | 9.2e2 | 1.2e3 | 3.1e1 |
| 264.16 | z₂ | — | 1.4e2 | 1.0e2 | — |
| 242.15 | z₂-COOH + Na | — | 6.2e2 | 1.9e3 | 1.1e2 |
| 238.11 | c₂ | — | 9.4e2 | 1.0e3 | — |
| 235.18 | y₂-COOH + H | — | 3.8e2 | 3.5e2 | 3.3e1 |
| 220.17 | z₂-COOH + H | 1.5e1 | 8.5e2 | 1.2e3 | 1.8e2 |
| 218.15 | z₂-COOH + | — | 1.9e2 | 2.0e2 | 7.0e1 |
| | H₂ + H | | | | |
| 193.10 | a₂ | — | 1.4e2 | 1.5e2 | 3.0e1 |
| 122.10 | Phe-COOH + H | 9.0e0 | 4.3e1 | 3.0e1 | 1.8e1 |
| 120.08 | Phe-COOH—H₂ + | 2.1e1 | 6.0e2 | 4.2e2 | 6.5e1 |
| | H | | | | |

Subsequent experiments on a range of standards, including bradykinin (Table 2), mNeonGreen (Table 3), ubiquitin, L-phenylalanine, DL-beta-phenylalanine, and creatinine also demonstrated the production of peaks unique to those compounds after exposure to the RF-AP source.

TABLE 2
Notable fragments, their annotations, and their
absolute intensities from direct infusion analysis
of bradykinin exposed to the RF-AP source.

| Peak | Annotation | Control | 5 seconds | 30 seconds | 1 minute |
|---|---|---|---|---|---|
| 1060.57 | M + H | 5.1e5 | 4.3e3 | 9.0e2 | 5.1e2 |
| 530.79 | M + 2H [2+] | 1.3e6 | 3.1e5 | 1.0e5 | 5.4e5 |
| 594.34 | c₅ + Na | — | 7.9e2 | 3.8e2 | 1.6e3 |
| 572.33 | c₅ | 2.0e2 | 6.8e2 | 4.1e2 | 1.2e3 |
| 491.26 | z₄ | 1.6e2 | 5.0e2 | 2.9e2 | 7.1e2 |
| 452.25 | c₈ + H [2+] | — | 2.9e2 | 1.2e2 | 3.8e2 |
| 447.27 | z₄-COOH + H | — | 1.9e3 | 9.9e2 | 4.2e3 |
| 425.26 | c₄ | 2.0e2 | 3.5e2 | 3.3e2 | 7.2e2 |
| 378.71 | c₇ + H [2+] | 5.2e1 | 1.6e2 | 1.3e2 | 4.3e2 |
| 307.18 | z₂ | — | 1.2e2 | 2.7e1 | 1.9e2 |
| 297.66 | c₅ + Na [2+] | 1.4e1 | 2.6e1 | 1.9e1 | 4.6e1 |
| 286.67 | c₅ + H [2+] | 2.3e1 | 1.7e2 | 1.4e2 | 4.5e2 |
| 263.19 | z₂-COOH + H | — | 1.7e3 | 9.3e2 | 4.3e3 |

TABLE 3
Notable fragments and their absolute intensities from direct
infusion analysis of mNeonGreen exposed to the RF-AP source.

| Peak | Control | 5 seconds | 30 seconds | 1 minute |
|---|---|---|---|---|
| 502.32 | 5.7e1 | — | 8.3e1 | 1.2e1 |
| 413.26 | 6.5e2 | 1.8e3 | 1.1e3 | 3.3e3 |
| 395.28 | 1.3e2 | 1.8e2 | 2.9e2 | 9.4e2 |
| 248.08 | — | 2.3e2 | 9.7e1 | 1.5e2 |
| 234.10 | — | 4.4e2 | 2.6e2 | 2.6e2 |
| 177.08 | — | 1.5e3 | 8.7e2 | 1.0e3 |
| 163.06 | — | 2.0e3 | 9.5e2 | 1.0e3 |
| 153.08 | — | 7.2e2 | 2.3e2 | 2.6e2 |
| 149.08 | 1.7e2 | 2.3e3 | 1.9e3 | 2.5e3 |
| 135.07 | 7.6e2 | 1.2e5 | 6.0e4 | 6.6e4 |

The RF-AP setup was applied also to Thermofisher Expi293F cells expressing the mNeonGreen monomeric fluorescent protein. In this instance, transfected and control cells were washed three times with ammonium acetate, then lysed through repeated freeze-thaw cycles using a −80° C. freezer. The lysate was applied to microscope slides, exposed to the RF argon plasma for one minute, then dissolved and filtered before direct injection-ESI analysis. This analysis workflow revealed peaks which were present in the transfected cell lysate which had been exposed to the argon plasma, but were absent in the unexposed mNeonGreen cell lysate and in the exposed control cell lysate. These results were replicated by analysis of the exposed lysates via MALDI imaging, showing localised peak generation.

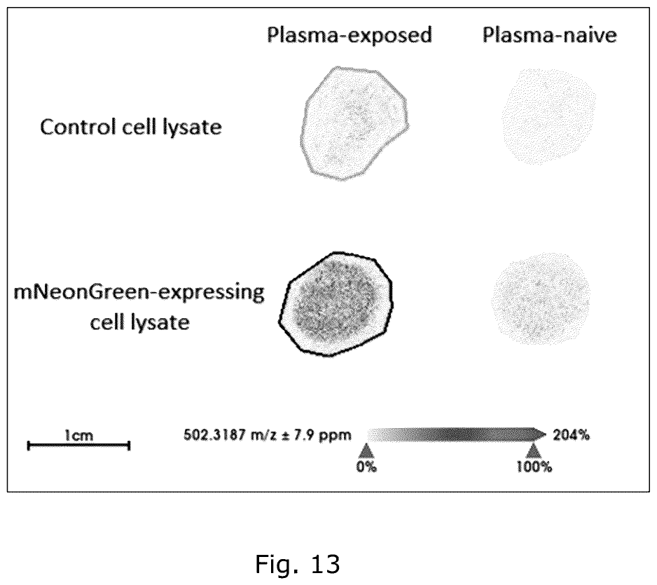

Finally, the argon plasma setup was applied to tissue samples. Four mouse kidney samples were washed twice for 30 seconds in 25 mL 70:30 ethanol:water, once for 15 seconds in 25 mL 100% ethanol, once for 10 seconds in 25 mL 90:9:1 ethanol:glacial acetic acid:water, and then twice for 2 seconds in 25 mL water. 1 uL of 1 ug/mL mNeonGreen standard was applied to two of the tissues and the RF argon plasma was applied to one control tissue and one spiked tissue for 1 minute each. 7 mg/mL CHCA was then applied to the slides as a matrix and the slides were analysed by MALDI. In this manner, we were able to differentiate plasma-exposed tissues from non-exposed tissues. See FIG. 13 which shows the resulting MALDI-MSI heatmap of a peak at 502.319 m/z in the lysates from the control cells and the cells modified to express the mNeonGreen fluorescent protein, where the lysate is either plasma-naïve or has been exposed to the cold argon plasma setup for one minute. This ion is present only above noise in the plasma-exposed cell lysate where mNeonGreen is present.

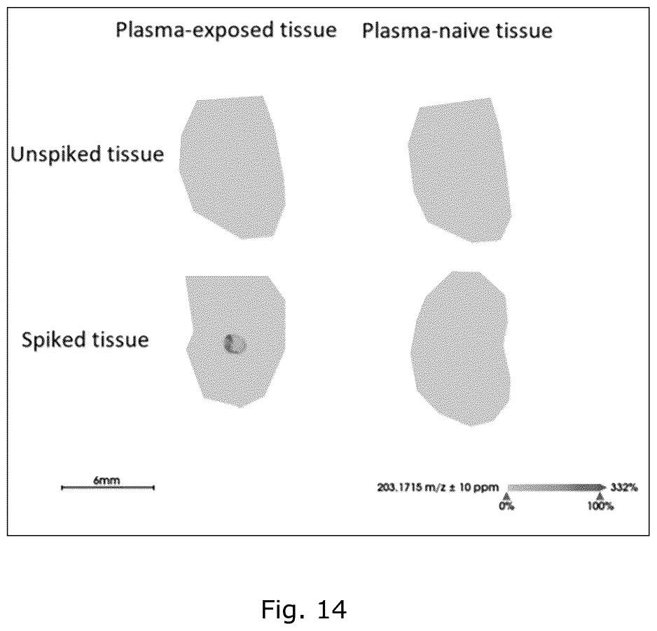

We were also able to differentiate mNeonGreen standard spiked onto a tissue sample from both exposed unspiked controls and unexposed spiked samples. Four intense and relevant peaks—distinguishable between the plasma-exposed standard and the controls—were noted at 158.11, 189.16, 203.17, and 204.17 m/z. The 204 m/z peak is likely the 13C isomer of the 203 m/z peak, which is singly charged. See FIG. 14 which shows the resulting MALDI-MSI heatmap of the peak at 203.172 m/z across the tissues which are optionally spiked and/or exposed to the cold argon plasma setup for one minute. This ion is present only above noise in the plasma-exposed tissue spiked with the fluorescent protein mNeonGreen

In conclusion, the invention provides a method of using an electrical discharge plasma or cold plasma, such as a cold argon plasma, for digestion of proteins. An important advantage of this approach over prior art methods is a substantial reduction in sample preparation time and effort and the elimination of reagents, including the use of enzymes. The use of electrical discharge plasmas and/or cold plasmas in this way also maintains the spatial integrity of the macromolecules without the delocalisation caused by liquid-phase digestions and enables high-throughput imaging of proteins in condensed phase samples.

Although particular embodiments have been described, a number of alternatives and variations will be apparent to the skilled person without departing from the scope of the invention for example as set out in the claims. For example, although mass spectrometry may particularly be used to detect the fragments so that the fragments, molecules and/or the sample can be characterised or identified, as noted above, using other analysis techniques and apparatus instead or in addition for these purposes such as nuclear magnetic resonance spectroscopy, and electron paramagnetic resonance spectroscopy, and various optical spectroscopy techniques.

1. A method of analysing organic molecules of a condensed phase sample, comprising:
generating a plasma using an electrical discharge;
exposing the condensed phase sample to the plasma so as to generate fragments of the organic molecules; and
detecting the fragments using a mass spectrometer.

2. The method of claim 1 wherein the condensed phase sample is located on a solid substrate while being exposed to the plasma.

3. The method of claim 1 wherein the condensed phase sample is a solid sample during exposure to the plasma to generate fragments of the organic molecules.

4. The method of claim 1 wherein the fragments of the organic molecules are generated within the condensed phase sample, and are liberated from the sample for detection by the mass spectrometer only after completion of the step of exposing the condensed phase sample to the plasma.

5. The method of claim 1 wherein at least a portion of the condensed phase sample is removed from the substrate following exposure to the plasma and introduced to the mass spectrometer for subsequent ionisation and detection of the generated fragments.

6. The method of claim 1 wherein the electrical discharge, the plasma, and the exposed sample are at one or more of: ambient atmospheric pressure, and between 30 and 300 kPa.

7. The method of claim 1 wherein the electrical discharge is generated by applying an electrical signal to one or more electrodes.

8. The method of claim 7 wherein the one or more electrodes comprise a plasma source electrode arranged to generate the plasma between the plasma source electrode and the sample, and a ground plane electrode disposed on an opposite side of the sample from the plasma source electrode, and optionally wherein the ground plane electrode is also separated from the sample by at least a portion of the substrate.

9. The method of claim 7 wherein the electrical signal is a DC signal.

10. The method of claim 1 wherein the electrical signal is an AC signal.

11. The method of claim 10 wherein the AC signal has a power of between 10 and 300 Watts and/or has a peak to peak voltage of between 1000 and 10000 V.

12. The method of claim 10 wherein the AC signal one or more of: is a radio frequency signal; and has a frequency of from 100 kHz to 5 MHz.

13. The method of claim 1 further comprising using a flow of a gas past the electrical discharge to the sample.

14. The method of claim 13 wherein the gas comprises at least 50%, or at least 90%, of one or more of: a noble gas, argon, helium, and nitrogen.

15. The method of claim 13, wherein the electrical discharge is generated by applying an electrical signal to one or more electrodes, and wherein at least one of the one or more electrodes is provided by a conductor extending along a conduit to an open end of the conduit, so that the electrical discharge takes place proximally to the end of the conduit, and the flow of the gas is along the conduit to the open end.

16. The method of claim 1 wherein the condensed phase sample is exposed to the plasma for one or more of: at least 1 second, at least 10 seconds, and at least 60 seconds.

17. The method of claim 1, wherein before the step of exposing the condensed phase sample to the plasma so as to generate fragments of the organic molecules, the method further comprises treating the condensed phase sample with a compound selected from a spin trap and a spin scavenger.

18. The method according to claim 17, wherein the compound is a spin trap, preferably wherein the spin trap is a nitrone or a nitroso compound, more preferably 5,5-dimethyl-1-pyrroline-N-oxide (DMPO) or N-tertiary-butyl nitrone (PBN).

19. The method according to claim 17, wherein the compound is a spin scavenger, preferably wherein the spin scavenger is an aminoxyl radical, more preferably (2,2,6,6-tetramethylpiperidin-1-yl)oxyl (TEMPO), 4-phosphonooxy-2,2,6,6-tetramethyl-1-piperidinyloxy 4-phosphonoxy-TEMPO (4-phosphonoxy-TEMPO) or 3-cyano-2,2,5,5-tetramethyl-1-pyrrolidinyloxyl (3-CP).

20. The method according to claim 17, wherein in the step of treating the condensed phase sample with a compound selected from a spin trap and a spin scavenger, the compound and the organic molecules are present in a weight ratio of from 0.1:1 to 10:1.

21. The method of claim 1 wherein the organic molecules comprise one or more of: proteins, peptides, lipids, and organic polymer molecules.

22. The method of claim 21 wherein the organic molecules are organic polymer molecules and the generated fragments of the organic molecules arise from fragmentation along the polymer backbone.

23. The method of claim 1, wherein detecting the fragments using a mass spectrometer comprises carrying out imaging mass spectrometry by detecting the fragments at multiple locations across the sample using an imaging mass spectrometer.

24. The method of claim 1 further comprising characterising or identifying the sample, the organic molecules, or the fragments using data from the mass spectrometer representing or including representation of the detected fragments.

25. Apparatus for analysing organic molecules of a condensed phase sample, comprising:
a plasma generator arranged to use an electrical discharge to generate a plasma;
a substrate carrier for receiving a substrate carrying the condensed phase sample for exposing the condensed phase sample to the generated plasma such that the plasma generates fragments of the organic molecules within the condensed phase sample; and
a mass spectrometer arranged to subsequently receive and detect the generated fragments.

26. The apparatus of claim 25 wherein the substrate is a solid substrate.

27. The apparatus of claim 25 arranged such that the mass spectrometer receives the generated fragments only after completion of exposure of the condensed phase sample to the generated plasma.

28. The apparatus of claim 25 further comprising a signal generator arranged to deliver an AC or DC electrical signal to the plasma generator for generating the plasma.

29. The apparatus of claim 28 wherein the electrical signal is an AC signal, and the AC signal one or more of: has a power of between 10 and 300 Watts; has a peak to peak voltage of between 1000 and 10000 V; and has a frequency of from 100 kHz to 5 MHz or is a radiofrequency signal.

30. The apparatus of claim 25 further arranged to provide a flow of a gas past the electrical discharge, and optionally to the sample.

31. The apparatus of claim 25 wherein the plasma generator comprises one or more electrodes to which the AC or DC signal is applied.

32. The apparatus of claim 31 wherein at least one of the one or more electrodes is provided by a conductor extending along a conduit to an open end of the conduit, so that the electrical discharge takes place proximally to the end of the conduit, and the flow of the gas is along the conduit.

33. The apparatus of claim 31 wherein the one or more electrodes comprise a plasma source electrode arranged to generate the plasma between the plasma source electrode and the sample, and a ground plane electrode disposed on an opposite side of the sample from the plasma source electrode.

34. The apparatus of claim 33 wherein the ground plane electrode is separated from the sample by at least a portion of the substrate.

35. The apparatus of claim 25 further comprising a sample conveyor mechanism arranged to carry some or all of the condensed phase sample to the mass spectrometer following treatment of the sample by the plasma.

36. The apparatus of claim 25 wherein the mass spectrometer is an imaging mass spectrometer.

37. The apparatus of claim 25 further comprising the condensed phase sample located on the substrate.

38. The apparatus of claim 25 wherein the organic molecules comprise one or more of: proteins, peptides, lipids, and organic polymer molecules.

39. The apparatus of claim 38 wherein the organic molecules are organic polymer molecules and the detected fragments of the organic molecules arise from fragmentation along the polymer backbone.

40. The apparatus of claim 25 arranged to characterise or identify the sample, the organic molecules, or the fragments, using data from the mass spectrometer representing the detected fragments.
